(19) United States
(12) Patent Application Publication
(10) Pub. No.: US 2021/0128367 A1
(43) Pub. Date: May 6, 2021

(54) CLOTHING MANUFACTURING APPARATUS AND CLOTHING MANUFACTURING METHOD

(71) Applicant: Kikuo YAMADA, Tokyo (JP)

(72) Inventor: Kikuo YAMADA, Tokyo (JP)

(21) Appl. No.: 16/090,774

(22) PCT Filed: Apr. 4, 2017
(86) PCT No.: PCT/JP2017/014110

§ 371 Date: Oct. 2, 2018

Related U.S. Application Data
(60) Provisional application No. 62/319,269, filed on Apr. 6, 2016.
(60) Provisional application No. 62/348,450, filed on Jun. 10, 2016.

(51) Int. Cl. A61F 13/15; A41H 43/02; D06G 3/02

(52) U.S. Cl. CPC A61F 13/15804; D06G 3/02; A41H 43/0264

(57) ABSTRACT

In order to provide a clothing manufacturing apparatus capable of improving production efficiency, a clothing manufacturing apparatus includes a cylindrical member having openings at both ends; and reversing means configured to reverse lower body clothing inside out by passing the lower body clothing in a state in which the cylindrical member is inserted through a waist opening of the lower body clothing and a crotch portion of the lower body clothing covers one of the openings of the cylindrical member through inside of the cylindrical member from the crotch portion.

### TECHNICAL FIELD

The present invention relates to a clothing manufacturing apparatus and a clothing manufacturing method.

### BACKGROUND ART

In manufacture of clothing such as pants and shirts, a back having joint portions such as sewn portions generally faces outside in a manufacturing phase Therefore, a reversing step for reversing inside out needs to be executed in order to prevent the joint portions from being exposed before shipment

Known in the related art is a technique to reverse a sleeve by inserting a pivotally provided arcuate shaped arm member from one of openings of the sleeve, catching the other opening of the sleeve with a tail portion of an arrowhead member provided at a distal end of the arm member, and in this state, pulling the arm member out from the sleeve (for example, see Patent Literature 1).

### CITATION LIST

### Patent Literature

Patent Literature 1: JP 11-81026 A

### SUMMARY OF INVENTION

### Technical Problem

However, since a technique described in Patent Literature 1 requires an operation to insert the arrowhead member and the arm member once to the clothing (sleeve) and then return backward again, it is time consuming and thus production efficiency is not good. Application of the technique described in Patent Literature 1 to clothing having three openings (for example, lower body clothing having a waist opening and two leg openings) is not easy.

It is an object of the present invention to provide a clothing manufacturing apparatus and a clothing manufacturing method having high production efficiency.

### Solution to Problem

A clothing manufacturing apparatus of the present invention includes: a cylindrical member having openings at both ends; and reversing means configured to reverse lower body clothing inside out by passing the lower body clothing in a state in which the cylindrical member is inserted through a waist opening of the lower body clothing and a crotch portion of the lower body clothing covers one of the openings of the cylindrical member through the cylindrical member from the crotch portion.

A clothing manufacturing method according to the present invention include: inserting a cylindrical member having openings at both ends into a waist opening of lower body clothing; setting the lower body clothing on the cylindrical member so as to cover one of the openings of the cylindrical member with a crotch portion of the lower body clothing; and reversing the lower body clothing inside out by passing the lower body clothing set on the cylindrical body through the cylindrical member from a crotch portion.

### Advantageous Effects of Invention

The clothing manufacturing apparatus and the clothing manufacturing method according to the present invention achieves an effect of improving production efficiency.

### BRIEF DESCRIPTION OF DRAWINGS

FIG. 1 is a drawing illustrating a cloth for a disposable pants of an embodiment.

FIG. 2 is a drawing illustrating a shirring part of the cloth for the disposable pants of FIG. 1.

FIG. 3 is an enlarged drawing illustrating the cloth for the disposable pants of FIG. 1.

FIG. 4 is a cross-sectional view taken along line A-A in FIG. 3.

FIG. 5 is a cross-sectional view taken along line B-B in FIG. 3.

FIG. 6 is a schematic drawing illustrating a manufacturing apparatus for disposable pants.

FIG. 7 is a flowchart illustrating a method of manufacturing the disposable pants.

FIG. 8(a) and FIG. 8(b) are drawings illustrating a first folding process.

FIG. 9(a) to FIG. 9(c) are drawings illustrating from a punching process to a joining process.

FIG. 10 is a drawing illustrating one piece of disposable pants obtained by cutting.

FIG. 11(a) to FIG. 11(d) are drawings illustrating a reversing device for disposable pants.

FIG. 12(a) to FIG. 12(d) are drawings illustrating a modification (1) of the reversing device for disposable pants.

FIG. 13(a) to FIG. 13(d) are drawings illustrating a modification (2) of the reversing device for disposable pants.

FIG. 14 is a drawing illustrating disposable pants attached with an absorbing member and worn.

FIG. 15 is a schematic drawing illustrating a modification of the disposable pants manufacturing apparatus.

FIG. 16 is a drawing illustrating part of the manufacturing apparatus illustrated in FIG. 15 viewed in a direction +Z.

FIG. 17(a) to FIG. 17(c) are drawings illustrating from a punching process to a joining process performed by the manufacturing apparatus in FIG. 15.

FIG. 18(a) and FIG. 18(b) are drawings illustrating the disposable pants after a cutting process performed by a modified disposable pants manufacturing apparatus.

### DESCRIPTION OF EMBODIMENT

A clothing manufacturing apparatus and a clothing manufacturing method of the present exemplary embodiment will be described below.

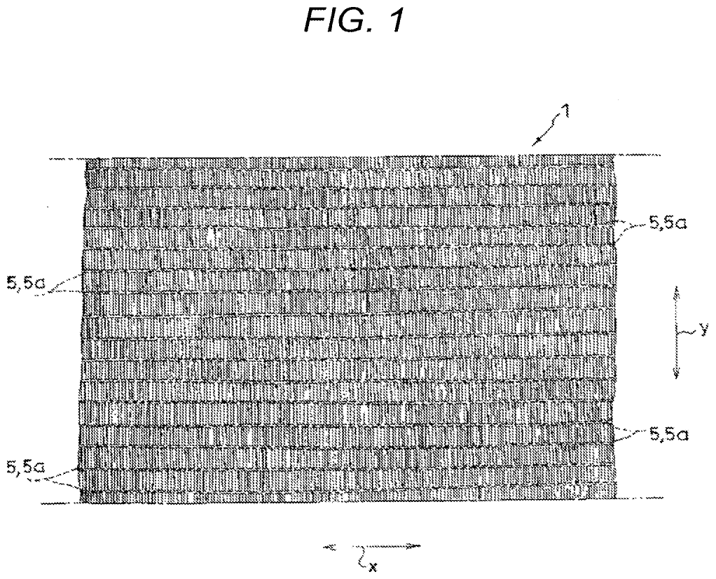

In the present exemplary embodiment, a cloth 1 illustrated in FIG. 1 is used as an outer sheet 11 of a disposable pants 10. The disposable pants 10 is a disposable lower body clothing. The cloth 1 may be applied to disposable diapers, and is also applicable as a surface material of an absorbing member 14 (absorbing pad) of the disposable diapers or disposable pants 10.

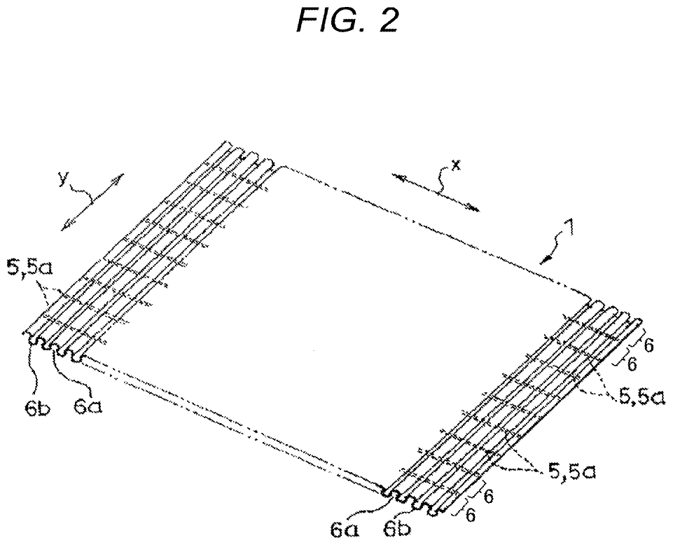

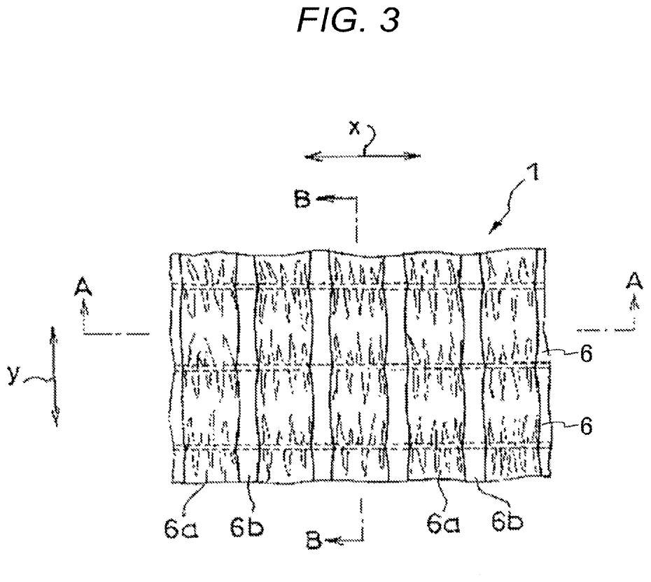

FIG. 1 is a drawing illustrating a surface state of the cloth 1 of the present exemplary embodiment, FIG. 2 is a drawing illustrating a shirring part of the cloth 1, and FIG. 3 is an enlarged view of the cloth 1. Referring now to FIG. 1 to FIG. 3, the cloth 1 will be described in detail.

In FIG. 1, the cloth 1 has a long configuration continuing in an x direction, which is a longitudinal direction (first direction).

A plurality of resilient members 5 are provided in an interior of the cloth 1 along the x direction and at predetermined intervals in a y direction, which is a short direction (second direction) of the cloth 1, as is understood from FIG. 1 and FIG. 2. The resilient members 5 expand and contract along the x direction. As illustrated in FIG. 2, an irregular surface, that is, a shirring part is formed on the cloth 1 by a number of resilient members 5.

Linear resilient members 5a having elasticity are used as the resilient members 5, and urethane, silicone, butadiene or styrene butadiene-based synthetic rubber, or natural rubber may be used.

Note that when a grating shaped resilient member is used, the above-described shirring part may be formed by a single grating shape. An elastic film may also be used instead of a number of the resilient members 5. Examples of the elastic film include urethane film, silicone film, and elastomer film.

In the following description, the linear resilient members 5a made of polyurethane is used.

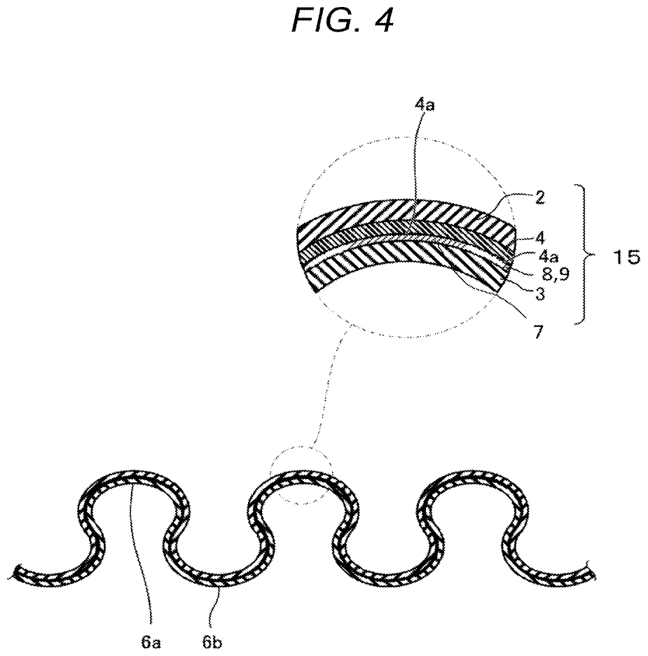

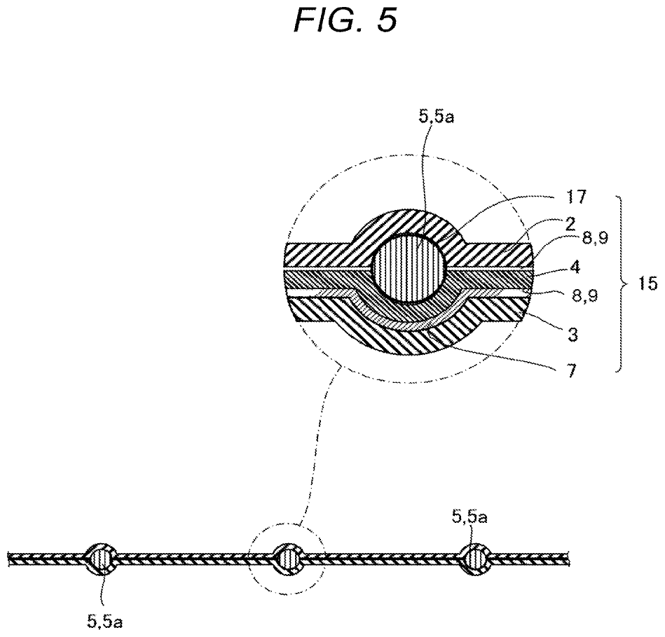

FIG. 4 is a cross-sectional view taken along line A-A in FIG. 3, and FIG. 5 is a cross-sectional view taken along line B-B in FIG. 3. As illustrated in FIG. 4 and FIG. 5, the cloth 1 is made of a multi-ply sheet 15 including a first fiber sheet 2 having air permeability, a second fiber sheet 3 having a liquid diffusion property, and a third fiber sheet 4 having a liquid diffusion property, being interposed between the first fiber sheet 2 and the second fiber sheet 3, and formed by layering the first fiber sheet 2, the second fiber sheet 3, and the third fiber sheet 4. The first fiber sheet 2 and the second fiber sheet 3 are made of a fiber layer having air permeability, and the third fiber sheet 4 is made of a fiber layer having liquid diffusion property. In this manner, the multi-ply sheet 15 includes three layers. The multi-ply sheet 15 may be a two-layer configuration by omitting the third fiber sheet 4, or may be a two-layer configuration by omitting the second fiber sheet 3.

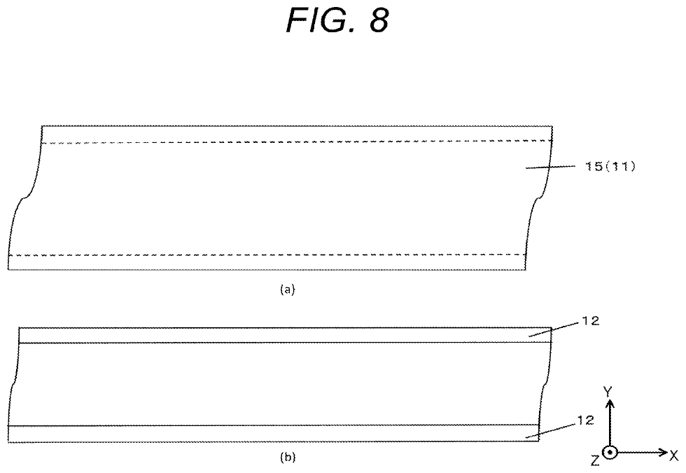

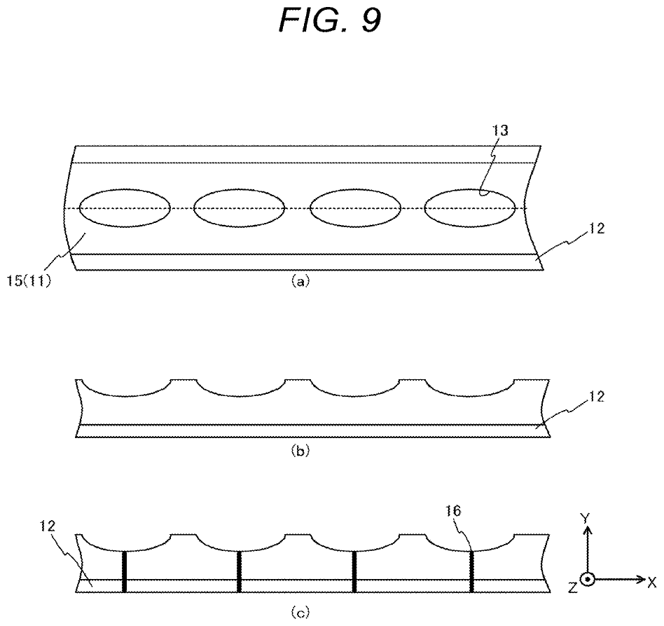

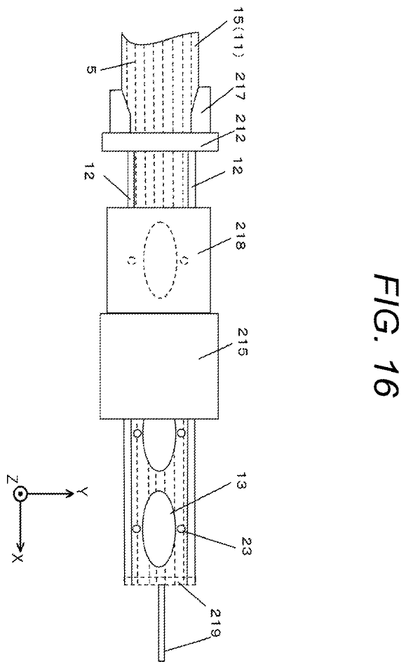

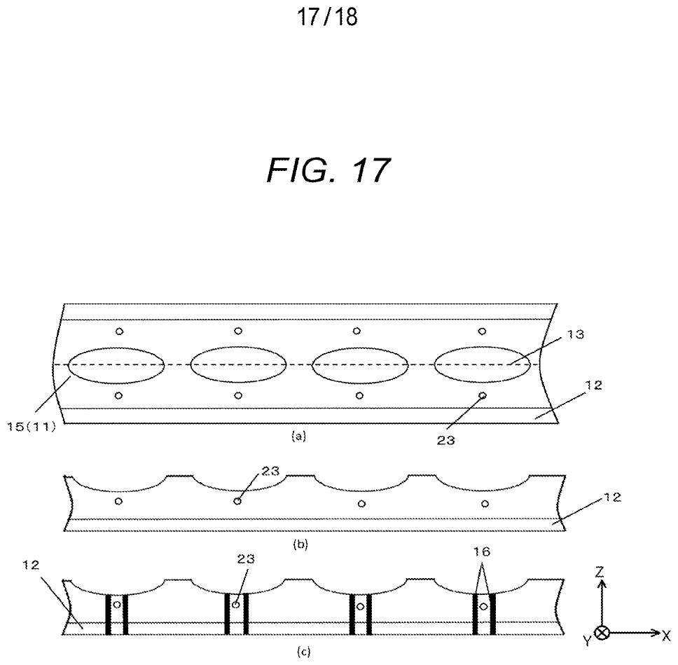

In the present exemplary embodiment, when the cloth 1 is used for the outer sheet 11 for the disposable pants 10 as described later, the first fiber sheet 2 faces a side that makes contact with a skin of a user (skin side), and the second fiber sheet 3 faces outside (non-skin side). The cloth 1 is applied to the outer sheet 11 so that an x direction of the cloth 1 in FIG. 1 matches an X direction in FIG. 8, FIG. 9, FIG. 16 and FIG. 17, and a y direction in FIG. 1 matches a Y direction in FIG. 8, FIG. 9, FIG. 16, and FIG. 17.

The first fiber sheet 2 is a non-woven fabric, and two-layer or three-layer spun bonded nonwoven fabric may be used. In the present exemplary embodiment, the first fiber sheet 2 of the cloth 1 is used for the skin side of the outer sheet 11 of the disposable pants 10. Therefore, a hydrophilic nonwoven fabric is preferably used as the first fiber sheet 2. When hydrophilic property is required for the cloth 1, for example, a hydrophilizing process such as adding a hydrophilizing agent to the cloth 1 may be performed.

Note that the basis weight of the first fiber sheet 2 is preferably 10 to 50 g/m2 and more preferably 10 to 20 g/m2 from a viewpoint of a direct cost of manufacture. However, the invention is not limited thereto.

The second fiber sheet 3 is a nonwoven fabric, and two-layer or three-layer spun bonded nonwoven fabric may be used. In the present exemplary embodiment, the second fiber sheet 3 is used for the outer (non-skin) side of the outer sheet 11 of the disposable pants 10, and thus a water repellent sheet may be employed. A three-layer spun bonded nonwoven fabric may be used as the water repellent nonwoven fabric, and water repellent treatment for coating fluorine-based, silicone-based, paraffin-metal-based, alkylchromic-chloride-based water repellant is preferably performed. Note that the basis weight of the water repellent nonwoven fabric is preferably 10 to 50 g/m2 and more preferably 10 to 20 g/m2 from a viewpoint of a direct cost of manufacture. However, the invention is not limited thereto.

A material made of pulp as a main material, that is, a material containing a cellulose-based component, may be used for the third fiber sheet 4, and a pulp fiber sheet such as tissue paper, crape paper, and air laid nonwoven fabric may be used. Examples of the pulp include wood pulp, synthetic pulp, and used paper pulp. The material is not limited to natural fibers such as the pulp, recycled fibers such as rayon may also be used. The basis weight of the third fiber sheet 4 is preferably 10 to 50 g/m2 for example. When the pulp fiber sheet is used, for example, embossing is preferably performed for providing the pulp fiber sheet with softness. When the cloth 1 is used for the outer sheet 11 of the disposable pants 10, a printing layer 4a may be formed on the third fiber sheet 4 (pulp fiber sheet) in advance for various printing.

As illustrated in FIG. 5, in the present exemplary embodiment, the first fiber sheet 2 and the third fiber sheet 4 are joined by an adhesive agent 17 applied over a peripheral surface of the resilient members 5. Therefore, in a portion where no resilient member 5 is present, a non-adhered portion 8 not applied with the adhesive agent 17 is present between the first fiber sheet 2 and the third fiber sheet 4, and the non-adhered portion 8 forms a space 9. In the present exemplary embodiment, since the space 9 is formed by the non-adhered portion 8, various functions including a moisture evaporation property, a heat radiation property, and a moisture permeability of the cloth 1 may be improved.

Instead of adhesion via the resilient members 5, or in addition to the adhesion via the resilient members 5, the adhesive agent 17 may be applied partly (intermittently) on at least one of the first fiber sheet 2 and the third fiber sheet 4 to join the first fiber sheet 2 and the third fiber sheet 4. In this case as well, since joining between the first fiber sheet 2 and the third fiber sheet 4 is effected partly (intermittently), the space 9 may be formed between the first fiber sheet 2 and the third fiber sheet 4.

Examples of the method of partly (intermittently) applying the adhesive agent 17 include applying in mist form, linear form, dotted form, stripe form, spiral form, block form, and pattern form. One of these forms may be employed or a plurality of forms may be combined.

Various types of adhesive agent such as a pressure sensitive adhesive agent and a curable adhesive agent may be used as the adhesive agent 17, and a hot melt adhesive agent is employed in the present exemplary embodiment. Note that the joint between the first fiber sheet 2 and the second fiber sheet 3 is not limited to the joint by adhesion, but various joining methods such as ultrasonic joining or heat sealing may be applied or may be used in parallel.

As illustrated in FIG. 2, FIG. 3, and FIG. 4, projections 6a and depressions 6b are continuously formed between the adjacent linear resilient members 5a, and thus the shirring part is formed entirely over the cloth 1.

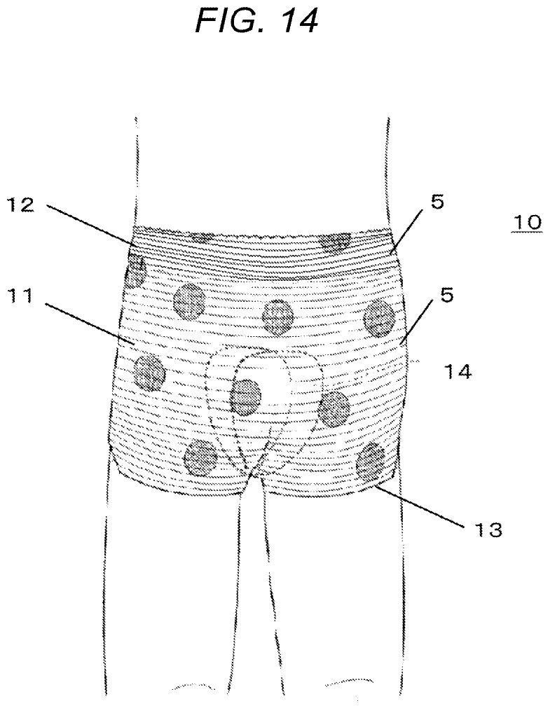

The intervals of the linear resilient members 5a in the y direction may be substantially uniform intervals and, for example, linear resilient members 5a may be arranged densely in the peripheral portion of the cloth 1 by shortening the intervals. In addition, the intervals may be shortened from a central portion to the peripheral portion of the cloth 1. Alternatively, or in parallel, a resilient force of the linear resilient members 5a in the peripheral portion of the cloth 1 may differentiated from a resilient force of the linear resilient members 5a in the center portion to enhance the resilient force of the linear resilient members 5a in the peripheral portion. In any cases, when the cloth 1 is used as the disposable pants 10, the linear resilient members 5a may be arranged so that the resilient force of a waist-worn portion 12 (see FIG. 14) becomes larger than the resilient force of other portions.

The linear resilient members 5a is provided between the first fiber sheet 2 and the third fiber sheet 4 as described before. A number of convex-concave rows 6 are formed in the y direction in FIG. 1 and FIG. 2. The number of the linear resilient members 5a per unit surface area may be set as appropriate. However, by forming many number of the linear resilient members 5a to narrow the mutual intervals of the linear resilient members 5a, the projections 6a and the depressions 6b in one convex-concave row 6 may be formed in a uniform shape, and in addition, may be maintained in shape. Accordingly, the shape of the shirring part is prevented from losing shape, and softness, a moisture transpiration property, a heat radiation property, and moisture permeability of the cloth 1 may be increased.

From such an objective, a pitch interval between the projections 6a in one convex-concave row 6 is preferably 2.00 mm to 7.00 mm. The pitch interval between the projections Ga is more preferably from 3.00 mm to 6.25 mm.

By narrowing the pitch interval between the projections Ga, a fine shirring is formed, and thus a good appearance is achieved. In addition, since the contact surface area with the skin per each fold of shirring is reduced, texture is improved. Furthermore, since the surface area is increased, absorbent property for absorbing sweat or the like is improved. In contrast, by widening the pitch interval between the projections 6a, the resilient force of the linear resilient members 5a may be reduced to some extent, and thus the manufacturing cost may be reduced.

Since the cloth 1 (also referred to as multi-ply sheet 15) has an elongated dimension, cutting is performed to make the longitudinal direction (the x direction in FIG. 1 and FIG. 2) of the multi-ply sheet 15 a predetermined length. In this cutting process, the first fiber sheet 2, the second fiber sheet 3, the third fiber sheet 4, and the resilient members 5 are cut. By cutting the resilient members 5, the resilient members 5 in the tensed state are released from the tension and contract by a restoring force. By the contraction stress applied at this time, the cloth 1 (multi-ply sheet 15) is subject to a force in the shortening direction. Therefore the cloth 1 (multi-ply sheet 15) is provided with the plurality of convex-concave rows 6, and thus the shirring part is formed. In this manner, the cloth 1 having a number of the shirring parts is manufactured.

Note that the cloth 1 may be changed in state from the expanded state to the contracted state by reducing a feeding speed of a feeding device (feeding roll), not illustrated, for feeding the cloth 1 before cutting.

The multi-ply sheet 15 is provided with a resilient force by the resilient members 5 disposed inside the multi-ply sheet 15. Therefore, when the cloth 1 made of the multi-ply sheet 15 is pulled in the x direction in FIG. 1 and FIG. 2, the cloth 1 is expanded and spread by expansion of the resilient members 5. When this state is released, the resilient members 5 contracts by the restoring force of its own and the cloth 1 is restored to an original state correspondingly. Since the cloth 1 has elasticity in this manner, the cloth 1 provides a superior fit with respect to the body when the cloth 1 is used as the disposable pants 10.

The dimensions of the disposable pants 10 is determined with the cloth 1 in the contracted state achieved by the restoring force of the resilient members 5, and thus the disposable pants 10 has a large surface area per unit volume. Therefore, even when a fluid of the body (including urine, sweat, blood, lymph fluid) leaks from an absorbing member 14 (see FIG. 14) having crushed pulp and Super Absorbent Polymer (hereinafter, referred to as SAP), which is a super absorbent resin in a form of particles, the fluid of body may be absorbed by the cloth 1 and probability of soiling an outer (trousers, pants) is eliminated. Note that the absorbing member 14 may be modified in various manner such as forming only of the SAP. In addition, the absorbing member 14 may be provided with a surface material (top sheet) made of the cloth 1.

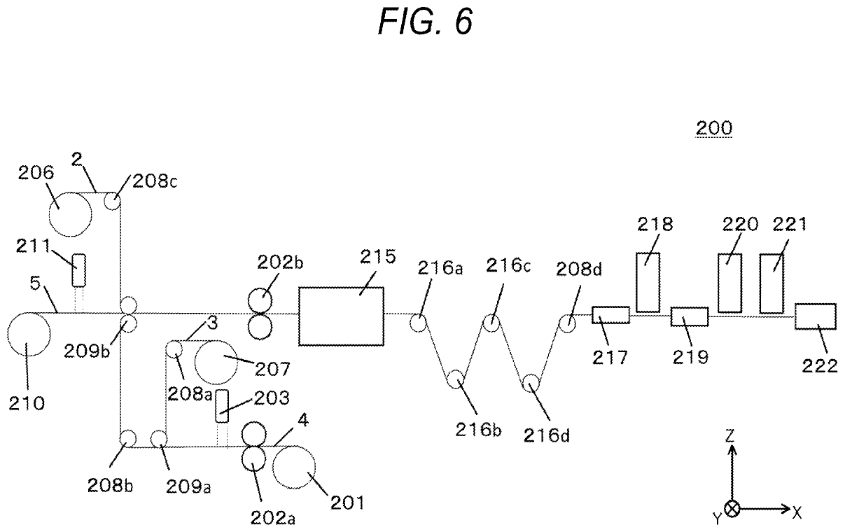

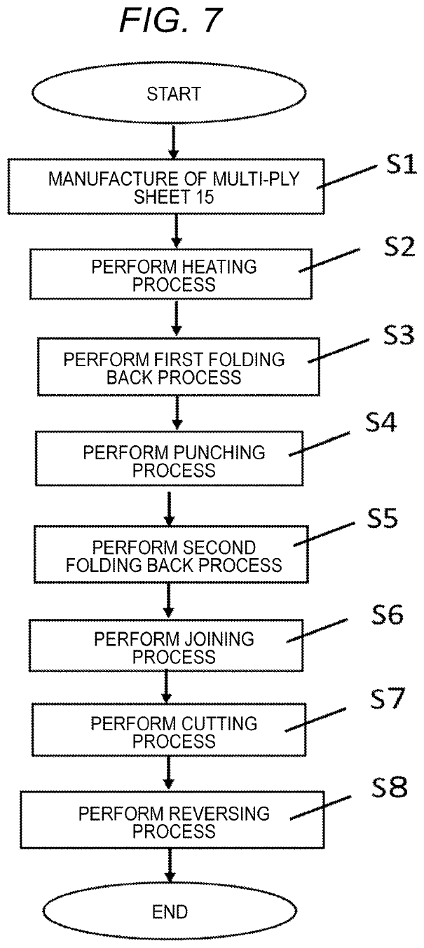

FIG. 6 is a schematic drawing illustrating a manufacturing apparatus 200 of the disposable pants 10, and FIG. 7 is a flowchart illustrating a method of manufacturing the disposable pants 10. A layout of the manufacturing apparatus 200 in FIG. 6 is an example only, and positions of installation of various devices may be modified if appropriate according to space conditions in factories.

(Step S1: Manufacture of Multi-Ply Sheet 15)

The third fiber sheet 4 rolled on a sheet roll 201 is fed in a −X direction and is embossed by a pair of emboss rolls 202a. Accordingly, the third fiber sheet 4 is softened. Since a plurality of the emboss rolls 202 are used in the manufacturing apparatus 200, reference signs (alphabets) are added to discriminate these emboss rolls. The emboss rolls 202 may be the same emboss rolls, or may be emboss rolls different in emboss pattern, size, or material. The third fiber sheet 4 wound around the sheet roll 201 has a pattern (see FIG. 14) printed in advance, and the third fiber sheet 4 with printed pattern is embossed as described above. When the cloth 1 having the third fiber sheet 4 as a surface material of the absorbing member 14 described above as well, printing may be performed on a printing layer 4a of the third fiber sheet 4. For example, the disposable pants 10 may be printed with words such as “Front” and “Back”, arrows, patterns, pictograms to provide a guide for attaching the absorbing member 14 to the disposable pants 10, or may be printed with notes for using the absorbing member 14, or even may be colored or printed with patterns. Printing on the third fiber sheet 4 is preferably performed on the third fiber sheet 4 before layering the cloth 1.

A first adhesive agent application device 203 applies an adhesive agent 7 to the third fiber sheet 4 that has passed through the emboss rolls 202a. The present exemplary embodiment employs a hot melt adhesive agent as the adhesive agent 7, but the adhesive agent 7 is not limited thereto. Note that the adhesive agent 7 is heated to a predetermined temperature (for example, to a range from 60° C. to 150° C.) by the first adhesive agent application device 203.

The second fiber sheet 3 wound around the sheet roll 207 is fed in a direction −Z by a feeding roll 208a and is joined to the third fiber sheet 4 applied with the adhesive agent 7 by pressure roll 209a. The second fiber sheet 3 and the third fiber sheet 4 joined together are fed in the direction +Z by a feeding roll 208b. Since the plurality of feeding rolls 208 are used in the manufacturing apparatus 200, reference signs (alphabets) are added for the purpose of discrimination in the description. However, the feeding rolls 208 may be rolls of the same configuration or may be feeding rolls different in size of rotating speed. In addition, since the plurality of pressure rolls 209 are used in the manufacturing apparatus 200, reference signs (alphabets) are added for the purpose of discrimination in the description. However, the pressure rolls 209 may be rolls 209 of the same configuration or may be different pressure rolls. The number of the pressure rolls 209 or the size and the pressing force of the pressure rolls 209 may be set as appropriate.

A second adhesive agent application apparatus 211 applies the adhesive agent 17 to the resilient members 5 wound around the resilient roll 210. The present exemplary embodiment employs a hot melt adhesive agent as the adhesive agent 17, but the adhesive agent 17 is not limited thereto. The adhesive agent 17 and the adhesive agent 7 may be the same hot melt adhesive agent, or may be different (for example, different in viscosity). Note that the adhesive agents 7, 17 are heated to a predetermined temperature (for example, to a range from 60° C. to 150° C.) by the second adhesive agent application device 211. In order to simplify the drawing, only one resilient roll 210 is illustrated. However, a plurality of the resilient rolls 210 are disposed depending on the number of resilient members 5 to be used.

The first fiber sheet 2 wound around the sheet roll 206 is fed in the direction −Z by a feeding roll 208c. In contrast, as described above, the second fiber sheet 3 and the third fiber sheet 4 joined together are fed in the direction +Z by a feeding roll 208b. A pair of the pressure rolls 209b press and join the first fiber sheet 2 to the second fiber sheet 3 and the third fiber sheet 4 joined together in advance via the resilient members 5 applied with the adhesive agent 17. Accordingly, the multi-ply sheet 15 is manufactured. The multi-ply sheet 15 is manufacturing by layering the first fiber sheet 2, the resilient members 5, the third fiber sheet 4, and the second fiber sheet 3 from the top (+Z side) in this order.

The multi-ply sheet 15 applies an emboss roll by the pair of emboss rolls 202b. Accordingly, the multi-ply sheet 15 is softened. Note that the pair of emboss rolls 202b may be provided on the downstream side (+X side) of a heating device 215, or may be provided on the upstream and downstream sides (−X side and +X side) of the heating device 215. The emboss rolls 202b may be omitted.

(Step S2: Heating Process)

The multi-ply sheet 15 is provided with a resilient force by the resilient members 5 disposed inside the multi-ply sheet 15 as described above. The surface area per unit length in the X direction may be increased to an extent about 1.5 times to 5 times by selecting the resilient force of the resilient members 5. For example, assuming that the dimension required in the X direction is 800 mm, 1200 mm to 4000 mm of the cloth 1 (expanded state) is used. In this case, assuming that a rate of contraction, which is a rate of contraction from a state expanded to a predetermined length to a state contracted to the maximum (catalogue value or theoretical value) by the restoring force of the resilient members 5 is ⅓, 2400 mm obtained by multiplying an inverse number of the rate of contraction of the resilient members 5 is required as the dimension of the cloth 1 in the X direction. However, there is a case where the rate of contraction becomes ½ even when the expanded state is released by performing cutting after the first fiber sheet 2, the second fiber sheet 3, and the third fiber sheet 4 are actually joined via the resilient members 5 or by reducing the feeding speed of the feeding roll. This seems to be because of influences of a temperature or moisture problem in the manufacturing factory, especially a low temperature under no temperature control, a problem of the resilient members 5 themselves (for example, residual strain of the resilient members 5), deterioration of contraction of the resilient members 5 due to curing of the adhesive agent 17 applied to the resilient members 5, and rigidity of the third fiber sheet 4. In addition, conditions of application of the adhesive agent 17 (application temperature, amount of temperature, viscosity, etc.) may also become influencers Likewise, conditions of application of the adhesive agent 7 (application temperature, amount of temperature, viscosity, etc.) may also become influencers.

Accordingly, in the present exemplary embodiment, the rate of contraction of the resilient members 5 is brought to be closer to the theoretical value by hardening the resilient members 5 through the heating process on the multi-ply sheet 15 when the resilient members 5 is of a heat hardening type. In the heating process, the multi-ply sheet 15 is fed in the expanded state.

The heating device 215 is a non-contact heating device configured to supply air, for example, at 30° C. to 80° C.

The heating roll 216 is heated to, for example, a temperature ranging from 30° C. to 80° C. The heating rolls 216a and 216c come into contact with the second fiber sheet 3 of the multi-ply sheet 15 and heat the multi-ply sheet 15, and the heating rolls 216b and 216d come into contact with the first fiber sheet 2 of the multi-ply sheet 15 to heat the multi-ply sheet 15. Note that although the four heating rolls 216 are used in the present exemplary embodiment, the number of the heating rolls is not limited and may be set if appropriate.

Note that although both of non-contact heating by the heating device 215 and contact heating by the heating rolls 216 are performed as the heating process in the present exemplary embodiment, either one may be omitted.

(Step S3: First Folding Process)

The multi-ply sheet 15 after subjected to the heating process is fed in the +X direction by the feeding roll 208d, and is folded back at both ends in the Y direction by a predetermined lengths by a first folding device 217 to form a waist-worn portion 12 (see FIG. 14) of the disposable pants 10. FIG. 8 is a drawing illustrating a first folding process. The multi-ply sheet 15, which corresponds to the outer sheet 11, is valley folded along a dotted line on the +Y side and a dotted line on the −Y side in FIG. 8(a) into the shape illustrated in FIG. 8(b). The folded portions correspond to the waist-worn portion 12 of the disposable pants 10.

The first folding device 217 is of a known type having a folding plate for folding back the multi-ply sheet 15 and a pressing portion for pressing the multi-ply sheet 15 folded back. The folded portions are joined by various joining methods such as a hot seal, a hot melt adhesive agent, or an ultrasonic joining. The first folding device 217 and the joining device, not illustrated, may be provided between the pair of pressure rolls 209b and the heating device 215. In this case, the first folding device 217 and the joining device may be provided either on the upstream side of the emboss rolls 202b or on the downstream side of the emboss rolls 202b.

(Step S4: Punching Process)

The multi-ply sheet 15 after subjected to the first folding process by the first folding device 217 is fed in the +X direction and is subjected to punching by a punching device 218 for forming leg portions 13 (see FIG. 14) of the disposable pants 10 by the punching device 218. The punching device 218 may be, for example, a die-cut roll, but is not limited thereto.

FIG. 9(a) to FIG. 9(c) are drawings illustrating from a punching process to a joining process. FIG. 9(a) illustrates the multi-ply sheet 15 after subjected to punching. Holes for legs (leg portions 13) are formed in the multi-ply sheet 15, and a portion between the adjacent two holes (leg portions 13) corresponds to a crotch portion. The punching process may be performed prior to the first folding process.

In the state illustrated in FIG. 9(a), the near side of the paper plane is the first fiber sheet 2, that is, the skin side as described above, and from then onward, the resilient members 5, the third fiber sheet 4, and the second fiber sheet 3 are layered in this order.

(Step S5: Second Folding Process)

The multi-ply sheet 15 has a longitudinal direction in the X direction and the short direction in the Y direction. In the second folding process, the second folding device 219 mountain-folds the multi-ply sheet 15 in a short direction along a central dotted line in FIG. 9(a) for the joining process in Step S6. The second folding device 219 includes an arm member configured to lift the multi-ply sheet 15 along at the central dotted line for mountain folding, and a drive unit configured to drive the arm member so that the multi-ply sheet 15 after having been mountain-folded is set in the feeding device, not illustrated. FIG. 9(b) illustrates the multi-ply sheet 15 folded back in the short direction along the central dotted line in FIG. 9(a). The multi-ply sheet 15 is in the mounted folded state in the state illustrated in FIG. 9(b).

Note that the second folding process may be performed by reversing the front and back of the multi-ply sheet 15 by a reversing mechanism, not illustrated, and valley folding the central portion of the reversed multi-ply sheet instead of mountain folding the central dotted line of the multi-ply sheet 15.

(Step S6: Joining Process)

The multi-ply sheet 15 mountain-folded in the short direction is then subjected to joint along the Y direction by the joining device 220 for forming the disposable pants 10. In the present exemplary embodiment, the joining device 220 is an ultrasonic joining device. However, the joining device 220 is not limited thereto, and may be a device configured to perform joining by various joining methods such as the hot seal and the hot melt adhesive agent.

FIG. 9(c) illustrates the multi-ply sheet 15 joined by the joining device 220. A plurality of joint portions 16 (side seals) are formed on the multi-ply sheet 15 along the Y direction. As is apparent from FIG. 9(c), in the present exemplary embodiment, the disposable pants 10 is reversed (the first fiber sheet 2 on the skin side is exposed outward), and the joint portions 16 is formed on the non-skin side.

(Step S7: Cutting Process)

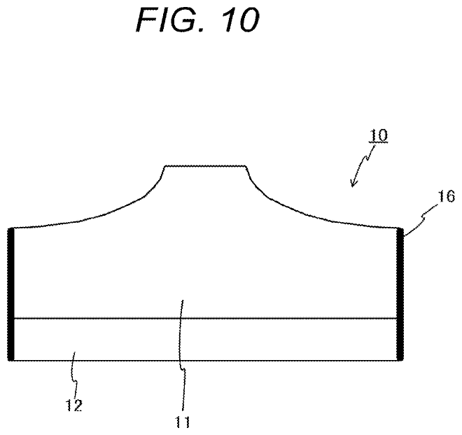

The multi-ply sheet 15 provided with the joint portions 16 is cut by a cutting device 221 provided with a cutter. In other words, the cutting device 221 cuts the joint portions 16 along the Y direction. Accordingly, one disposable pants 10 is manufactured. Since the state illustrated in FIG. 10 is a state in which the disposable pants 10 is reversed (the state in which the first fiber sheet 2 on the skin side is exposed outward), a process to bring the second fiber sheet 3 on the side opposite from the skin side to the front surface (reversing process) needs to be performed to make the disposable pants 10 ready to wear.

(Step S8: Reversing Process)

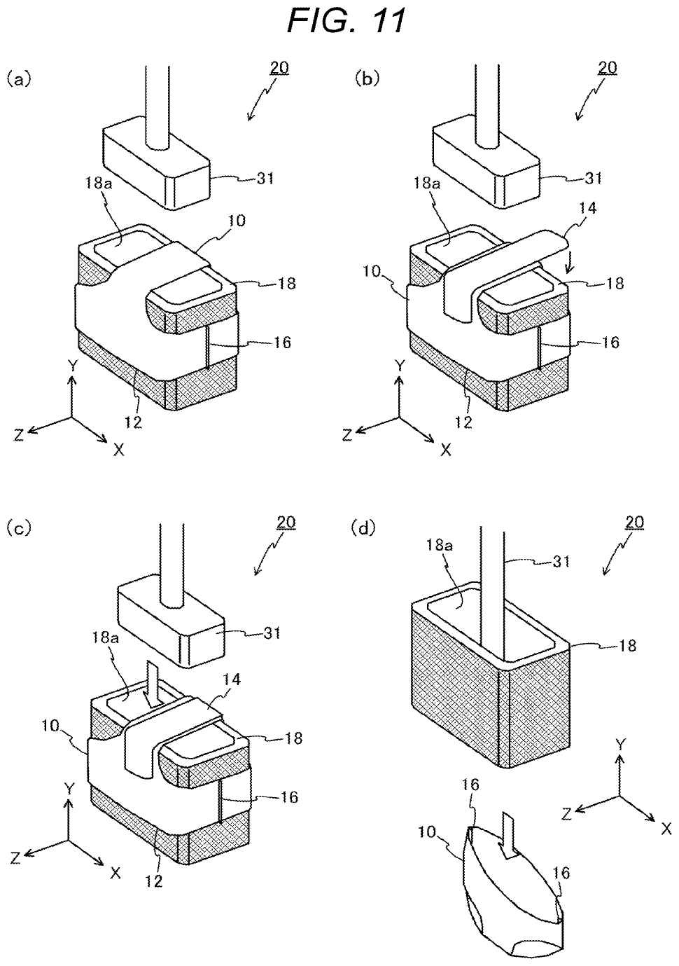

FIG. 11(a) to FIG. 11(d) are drawings illustrating a reversing device 20 for the disposable pants 10. The reversing device 20 includes a cylindrical member 18 and a pushing device 31.

As illustrated in FIG. 11(a), the cylindrical member 18 has a shape of a substantially parallelepiped and includes a through hole 18a from the +Y side surface to a −Y side surface. In other words, the cylindrical member 18 includes openings on the +Y side surface and the −Y side surface. However, the cylindrical member 18 does not necessarily have to have a shape of the substantially parallelepiped, and may have a cylindrical shape, an oval cylindrical shape, or the cylindrical shape and the oval cylindrical shape may be tapered. The cylindrical member 18 may have an opening or may be cut out partly on a side wall as long as the entire shape has a cylindrical shape.

An outer surface of the cylindrical member 18 (specifically a surface except for the +Y side surface and −Y side surface) has a rough surface by providing a mat finish for generating resistance against the movement of the disposable pants 10. The internal surface of the through hole 18a has a smooth surface not to provide any resistance against the movement of the disposable pants 10.

The pushing device 31 move reciprocally in the Y direction by a drive device, not illustrated. The movement in the Y direction cause the pushing device 31 to enter the through hole 18a of the cylindrical member 18 or come out upward from the through hole 18a.

Subsequently, an operation of the reversing device 20 will be described.

As illustrated in FIG. 11(a), the cylindrical member 18 is inserted into the disposable pants 10 through the waist-worn portion 12 by a device, not illustrated, or manually from the +Y side. Note that the length of an outer periphery of the cylindrical member 18 matches the length of an inner periphery of the waist-worn portion 12 (inner periphery of the waist opening) when the waist-worn portion 12 is stretched against the resilient force of the waist-worn portion 12. Therefore, in the state of FIG. 11(a), the disposable pants 10 is retained by the cylindrical member 18 with a suitable retaining force by the resilient force of the waist-worn portion 12. In the state illustrated in FIG. 11(a), the crotch portion of the disposable pants 10 covers part of the through hole 18a (opening on the +Y side surface) of the cylindrical member 18 from the +Y side.

After the cylindrical member 18 is retained by the disposable pants 10, the absorbing member 14 is attached to the crotch portion of the disposable pants 10 as illustrated in FIG. 11(b) by a device, not illustrated or manually.

After the absorbing member 14 is attached, the pushing device 31 moves toward the crotch portion of the disposable pants 10 in the −Y direction as illustrated in FIG. 11(c), and passes in the through hole 18a of the cylindrical member 18 in a state of pushing the crotch portion. Accordingly, the disposable pants 10 is mechanically pushed by the pushing device 31, passes through the cylindrical member 18 (in the through hole 18a), and comes out from the through hole 18a as illustrated in FIG. 11(d). In this case, the disposable pants 10 is in a state of inside out, that is, in the reversed state. In the state illustrated in FIG. 11(d), the joint portions 16 are positioned inside (skin side). Therefore, when the user wears the disposable pants 10, the joint portions 16 faces the skin side, and thus the joint portions 16 are invisible from outside. Accordingly, fashionability of the disposable pants 10 is improved. In particular, a feeling of hesitation of users who feel challenged to wear paper diapers for light incontinentia or the like may be alleviated.

In the present exemplary embodiment, the disposable pants 10 is reversed by a simple method such as making the cylindrical member 18 to retain the disposable pants 10 and pushing the pushing device 31 into the through hole 18a of the cylindrical member 18, and also may be removed (separated) from the cylindrical member 18. Accordingly, reversing process may be performed efficiently, and thus productivity of the disposable pants 10 may be improved. In the present exemplary embodiment, since the outer surface of the cylindrical member 18 is a rough surface, a suitable resistance is applied when the disposable pants 10 moves along the outer surface of the cylindrical member 18. Accordingly, the disposable pants 10 can be reversed easily, and thus the disposable pants 10 may be reversed without fail.

In FIG. 11(a) to FIG. 11(d), a case where the pushing device 31 is used for pushing the disposable pants 10 into the through hole 18a has been described. For example, the disposable pants 10 may be pushed into the through hole 18a by using a fluid.

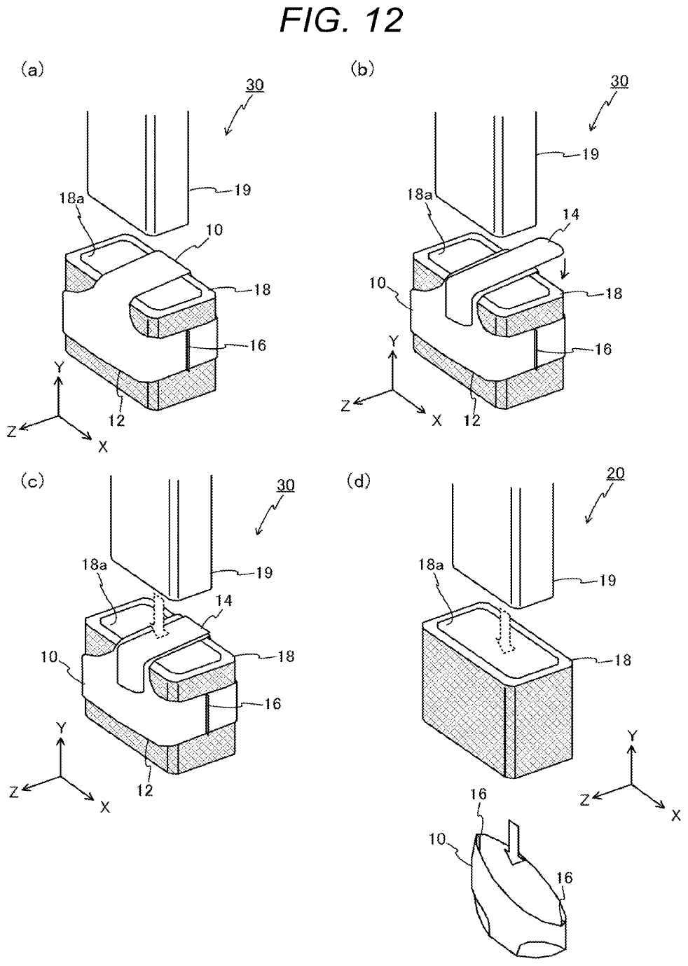

FIG. 12(a) to FIG. 12(d) are drawings illustrating an example using a fluid (reversing device 30). The reversing device 30 is provided with a fluid supply device 19 instead of the pushing device 31. The fluid supply device 19 is configured to supply a fluid compressed toward the crotch portion of the disposable pants 10, and for example, is configured to supply a compressed gas. Examples of the compressed gas include air having a pressure ranging from 0.2 to 1 MPa although various gases may be used.

In the reversing device 30, when the disposable pants 10 is retained on the is retained in the cylindrical member 18 and the absorbing member 14 is attached to the crotch portion as illustrated in FIG. 12(a) and FIG. 12(b), the fluid supply device 19 supplies compressed air toward the crotch portion of the disposable pants 10 as illustrated in FIG. 12(c) (see an arrow of a broken line in FIG. 12). A force in the −Y direction is applied to the crotch portion of the disposable pants 10, and the disposable pants 10 passes through the through hole 18a of the cylindrical member 18 and comes out from the through hole 18a of the cylindrical member 18 as illustrated in FIG. 12(d). In this case, the disposable pants 10 is in the reversed state. In the state in FIG. 12(d), the joint portions 16 faces inside (skin side).

Note that in the reversing device 30 illustrated in FIG. 12(a) to FIG. 12(d), the fluid supply device 19 may move in the −Y direction while supplying the compressed gas. Accordingly, the force in the −Y direction may be applied to the crotch portion of the disposable pants 10 efficiently.

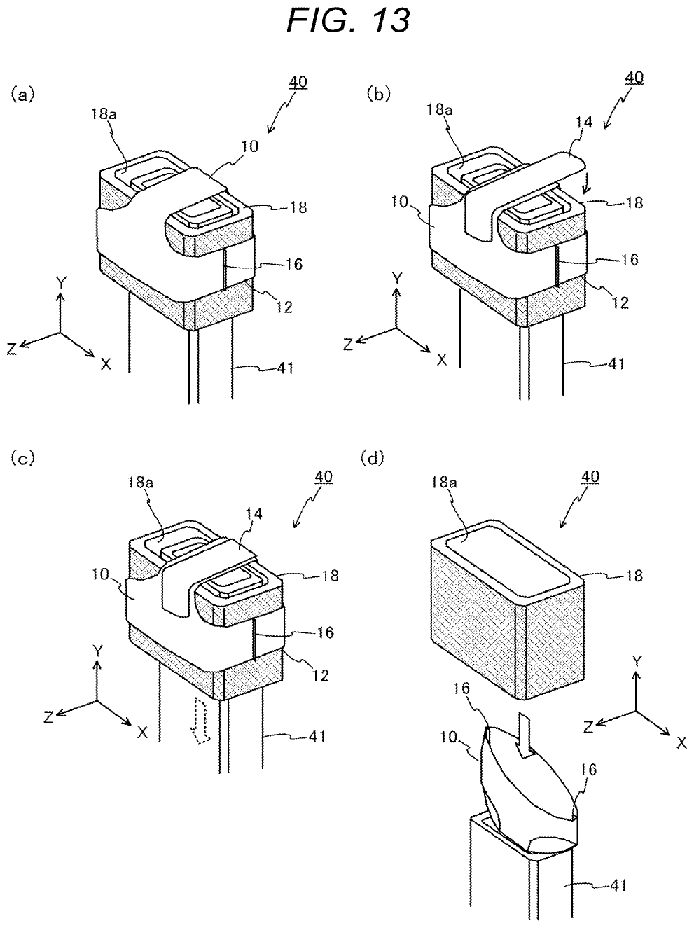

Note that a reversing device 40 provided with a suction device 41 capable of entering the through hole 18a of the cylindrical member 18 from the −Y side may be used instead of the pushing device 31 and the fluid supply device 19 as illustrated in FIG. 13(a) to FIG. 13(c). The suction device 41 is configured to suck air by driving a fan, not illustrated.

In this example, as illustrated in FIG. 13(a), the disposable pants 10 is retained in the cylindrical member 18 in a state in which the suction device 41 enters (is inserted into) the through hole 18a of the cylindrical member 18. As illustrated in FIG. 13(b), when the absorbing member 14 is attached to the crotch portion of the disposable pants 10, suction of the suction device 41 is started and the crotch portion is adsorbed as illustrated by an arrow in a broken line in FIG. 13(c). In this state, the suction device 41 starts movement toward the −Y side, and the disposable pants 10 passes through the through hole 18a and stops movement toward the −Y side when the disposable pants 10 comes out from the through hole 18a. In this case, the disposable pants 10 is in the reversed state as illustrated in FIG. 13(d), which is a state in which the joint portions 16 faces inside (skin side).

Note that the reversing devices 20, 30, and 40 may be combined if appropriate to constitute the reversing device. In the above-described reversing devices 20, 30, and 40, a pushing force, compressed air, or suction force is applied to the disposable pants 10. However, the disposable pants 10 may be reversed inside out (reversed) by applying other actions to the disposable pants 10. In the above-described reversing devices 20, 30 and 40, the absorbing member 14 is described to be attached to the crotch portion of the disposable pants 10. However, the absorbing member 14 does not necessarily have to be attached. In the manufacturing apparatus 200 in FIG. 6, the reversing device 20, 30, and 40 are illustrated as a reversing device 222.

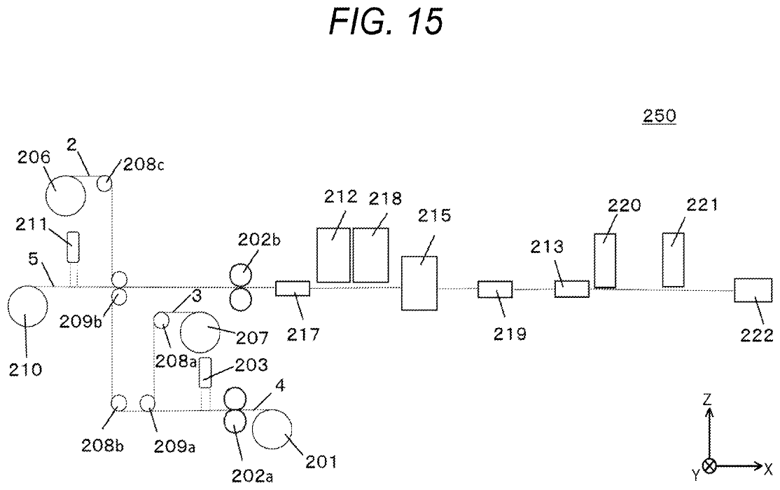

FIG. 15 is a schematic drawing illustrating the manufacturing apparatus 250 of the disposable pants 10 according to a modification and illustrates an example in which the layout and the manufacturing method of the manufacturing apparatus 200 in FIG. 6 is partly modified. Note that the same components as in the manufacturing apparatus 200 are denoted by the same reference signs, and description about overlapped portions will be omitted. A process until the multi-ply sheet 15 is passed through the pair of emboss rolls 202b is common to the manufacturing apparatus 200, and thus description will be omitted. Note that the multi-ply sheet 15 manufactured in a separate device may be set to the manufacturing apparatus 200 and the manufacturing apparatus 250, and the multi-ply sheet does not necessarily have to be manufactured in line.

The multi-ply sheet 15 passed through the pair of emboss rolls 202b is folded at an upper end (end on the +Y side) and at a lower end (end on the −Y side) by the first folding device 217. FIG. 16 is a drawing of part of the manufacturing apparatus 250 viewed in +Z direction and illustrates from the first folding device 217 to the second folding device 219. The first folding device 217 includes a folding plate for folding both end portions of the multi-ply sheet 15 in the Y direction, and the pressing portion configured to press the folded end portions. Note that in the manufacturing apparatus 250, the multi-ply sheet 15 is fed in a state of being expanded in the X direction except for the heating process on the downstream.

A joining device 212 is an apparatus configured to join the portion folded by the first folding device 217 along the X direction. The joining device 212 in the present exemplary embodiment is configured to perform ultrasonic joining. However, the joining device 212 may join by using an adhesive agent (hot melt adhesive agent or a heat seal) as described in conjunction with manufacturing apparatus 200. Accordingly, the waist-worn portion 12 of the disposable pants 10 may be formed.

The punching device 218 is a device for punching the multi-ply sheet 15 for forming the leg portions 13, and includes a Victoria blade (Thomson blade) as indicated by a dotted line in FIG. 16. The Victoria blade includes a blade for the leg portion 13 and blades for forming hole portions 23 at symmetrical positions about the hole for the leg portion 13 in the +Y side and the −Y side. The Victoria blade moving downward toward the multi-ply sheet 15 may form holes. The manufacturing apparatus 250 forms the leg portions 13 in the multi-ply sheet 15 and a pair of hole portions 23 for detecting the position of the multi-ply sheet 15. Note that one or three or more hole portion 23 may be formed. Instead of the Victoria blade, a die-cut roll may be used.

As described above, the multi-ply sheet 15 is fed by the feeding device, not illustrated in a state of being expanded in the X direction. For example, the rate of contraction of the resilient members 5 seems to be ⅓. In this case, when the multi-ply sheet 15 is fed in a state of being expanded to the maximum (most expanded state) by, for example, increasing the feeding speed, and the holes for the leg portions 13 are formed by the punching device 218, the multi-ply sheet 15 cannot be expanded more than the most expanded state, and thus the holes cannot increase in size anymore and can only decrease in size. In contrast, when the multi-ply sheet 15 is fed in a state of being expanded to an extent between the most expanded state and the maximum contracted state by, for example, decreasing the feeding speed, and the holes for the leg portions 13 are formed by punching device 218, the holes for the leg portions 13 increases in size when the multi-ply sheet 15 is expanded in the X direction, and the holes for the leg portions 13 decreases in size when the multi-ply sheet 15 contracts. Note that the same applies to the waist circumference of the disposable pants 10. The disposable pants 10 manufactured by feeding in the most expanded state has a smaller waist circumference, and the disposable pants 10 manufactured by feeding in the contracted state has a larger waist circumference. In this manner, in the manufacturing apparatus 250, the disposable pants 10 of a plurality of sizes may be manufactured by changing the expanded state when the multi-ply sheet 15 is fed.

In the manufacturing apparatus 250, the multi-ply sheet 15 is heated by the heating device 215 after the punching by the punching device 218. The heating device 215 is configured to supply heated air for improving the rate of contraction of the resilient members 5 as described above. The manufacturing apparatus 250 reduces the feeding speed by the feeding device, not illustrated, and supplies heated air to the multi-ply sheet 15 in a state in which the multi-ply sheet 15 is not expanded. The heating temperature and the heating period by the heating device 215, and whether the multi-ply sheet 15 is to be expanded may be determined experimentally. As described above, for example, the multi-ply sheet 15 is manufactured by a separate device and the rate of contraction of the multi-ply sheet 15 in the X direction has no problem, the heating device 215 may be omitted.

FIG. 17 is a drawing illustrating from a punching process to a joining process of the manufacturing apparatus 250. As is apparent from FIG. 17(a) to FIG. 16, the holes for the leg portions 13 and pairs of hole portions 23 for detecting the position are formed in the multi-ply sheet 15 passed through the heating device 215. A method of detecting the pairs of hole portions 23 will be described later. The second folding device 219 includes an arm member configured to lift the multi-ply sheet 15 for mountain fold and an acceptance and delivery member configured to accept and deliver the multi-ply sheet 15 from and to the arm member.

A hole detection device 213 illustrated in FIG. 15 is configured to detect the hole portions 23 (see FIG. 17(b)) of the multi-ply sheet 15 in the mountain folded state and output results of detection to the joining device 220. Each pair of hole portions 23 are substantially aligned in position in the X direction and the Z direction of the two hole portions 23 in the mountain folded state. In addition, by forming the hole portions 23 with the multi-ply sheet 15 expanded in the X direction, the pitches of the hole portions 23 in the X direction may also be uniformized. The hole detection device 213 is capable of detecting positions of the hole portions 23 with a non-contact manner by irradiating the hole portions 23 with detection light emitted from an irradiation part and receiving the detection light passed through the hole portions 23 with a light-receiving part. A configuration having one hole portion 23 and detecting the one hole portion 23 from a change of a light amount received by the light-receiving part is also applicable. A translucent type configured to receive detection light passed through the hole and a reflection type configured to receive reflected detection light both are applicable for the hole detection device 213. Instead of the system for detecting the hole portions 23, a system for detecting a mark is also applicable. In this case, for example, the punching process is replaced by a marking process, and in a marking process, the mark may be formed on the multi-ply sheet 15 for detection.

The joining device 220 is a device configured to join the multi-ply sheet 15 after the mountain fold along the Z direction. The joining device 220 in the present exemplary embodiment performs the ultrasonic joining. However, joining with hot melt adhesive agent or heat seal is also applicable. The joining device 220 joins left and right sides (+X side and −X side) of the hole portions 23 by ultrasonic joining as illustrated in FIG. 17(c) to form joint portions (side seals) 16 in response to the result of detection of the hole detection device 213.

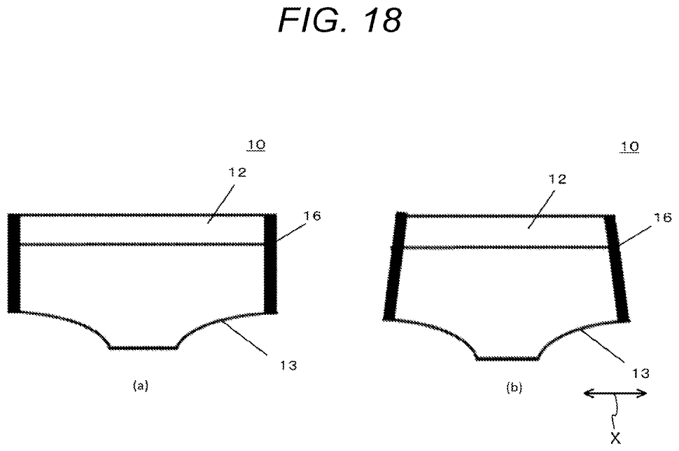

FIG. 17(c) illustrates a case where the joint portions 16 are two linear portions with the hole portions 23 in between. However, the shape of joint portions 16 may be set to have a shape different from those in FIG. 17(c) by changing the shape of anvil (receiving jig) of the joining device 220 into a V-shape or an inverted V-shape. For example, for the disposable pants 10 for women, the multi-ply sheet 15 may be joined by ultrasonic joining in a tapered shape widening from the waist portion (waist-worn portion 12) toward the seat and cut along the tapered shape by the cutting device 221 (see FIG. 18(b)).

In this manner, by selecting the shape of the joint portions 16 as appropriate, improvement in design of the disposable pants 10 is achieved. For the disposable pants 10 for women, the disposable pants 10 smaller in size in the X direction than those for men may be manufactured by feeding the multi-ply sheet 15 in a state in which they are longer than those for men as described above during various steps. Since the pitches of the hole portions 23 in the X direction are uniform in accuracy required for manufacture of the disposable pants 10 as described above, the joining device 220 may join portions in the vicinity of the hole portions 23 detected by the hole detection device 213, and may join portions in the vicinity of the hole portions 23 advanced by one pitch in the +X direction.

The cutting device 221 cuts portions (near the hole portions 23) interposed between portions (joint portions 16) joined by ultrasonic joining. Since the pitches of the hole portions 23 in the X direction are uniform, the portions in the vicinity of the hole portions 23 advanced by several pitches in the +X direction may be cut in response to the detection by the hole detection device 213. The hole detection device 213 may also be provided between the joining device 220 and the cutting device 221 and configured to perform cutting in response to the results of detection of the hole detection device 213.

FIG. 18 illustrates the disposable pants 10 after the cutting process. FIG. 18(a) illustrates a disposable pants 10 for men, and FIG. 18(b) illustrates a disposable pants 10 for women. As described above, the disposable pants 10 for women has a tapered shape, and the dimension in the X direction is smaller than that of the disposable pants 10 for men. The disposable pants 10 illustrated in FIG. 18 are inside out in the same manner as FIG. 10, and thus the reversing process by a reversing device 222 (reversing devices 20, 30, or 40) is performed as described above. In the manufacturing apparatus 250, manufacture of the disposable pants 10 is performed in this manner. In the exemplary embodiment described above, the manufacturing apparatus 200 and the manufacturing apparatus 250 have been described. However, various modifications may be made as a matter of course, and for example, the manufacturing apparatus 200 may be configured to form the hole portions in the multi-ply sheet 15 and provided with the hole detection device 213. In this manner, the manufacturing apparatus 200 and the manufacturing apparatus 250 may be combined if appropriate.

FIG. 14 is a drawing illustrating the disposable pants 10 attached with an absorbing member 14 and worn. Referring now to FIG. 14, the disposable pants 10 of the present exemplary embodiment will be described. In FIG. 14, although not illustrated, the absorbing member 14 is provided, for example, with adhesive tapes (at two positions, for example) on the skin side and the non-skin side on the opposite side to allow attachment to and removal from the disposable pants 10. Note that the absorbing member 14 may not be a removable tape, and may be a type fixed to the disposable pants 10.

The disposable pants 10 is made of the outer sheet 11, and part of the disposable pants 10 is provided with the waist-worn portion 12 to be worn around the waist of the user by folding the outer sheet 11.

In the present exemplary embodiment, the outer sheet 11 includes the first fiber sheet 2, the resilient members 5, the third fiber sheet 4, and the second fiber sheet 3 in sequence from the side coming into contact with the user's skin.

The waist-worn portion 12 is formed by bending the outer sheet 11 as described above, the resilient members 5 (the linear resilient members 5a) may be disposed densely compared with other portions, and the resilient force acting on the waist circumference of the user is stronger than other portion. Therefore, the disposable pants 10 is prevented from slipping down easily when the disposable pants 10 is worn. Note that the waist-worn portion 12 may be formed without bending the outer sheet 11.

The resilient members 5, being provided in a manner as to expand and contract in the lateral direction in FIG. 14, may also disposed in a manner as to expand and contract in the vertical direction in FIG. 14. As is apparent from FIG. 14, the disposable pants 10 is provided with a pattern. The pattern is printed to form the printing layer 4a on the third fiber sheet 4, and may be printed if appropriate by using a printing method such as gravure printing or a flexo printing.

The disposable pants 10 of the present exemplary embodiment is durable for washing for several times. Therefore, the disposable pants 10 can be used for a certain period of time by replacing the absorbing member 14, and thus is cost efficient.

The disposable pants 10 of the present exemplary embodiment may be used irrespective of ages, sexes, and physical structures (such as for kids, for men, and for women), and may also be used for animals such as pets.

Apart from the disposable pants 10, the cloth 1 of the present exemplary embodiment may be applied to clothing such as T-shirts and tank tops. In the clothing manufacturing method as well, the heating step and the reversing step as described above may be applied. For example, in the reversing step, the clothing may be reversed inside out by applying an action from the pushing device 31, the fluid supply device 19, and the suction device 41 in a state in which the clothing is retained by the cylindrical member 18 if the clothing is short clothing having no or short sleeve or no or short leg portions such as tank tops and short pants. The reversing step may be applied to clothing (such as pants) other than disposable closing. Accordingly, productivity of the clothing except for the disposable type may be improved.

Although the present exemplary embodiment has been described thus far, the invention is not limited thereto, and it is needless to say that various modifications or combinations if appropriate are possible. For example, the mode of arrangement of the linear resilient members 5a is not limited to the type extending linearly in the X direction. Therefore, intermittent linear resilient members 5a are applicable, bowed and curved linear resilient members 5a may be arranged in parallel, and wavy curved linear resilient members 5a may be arranged irregularly. In the arrangement of a number of the linear resilient members 5a, the respective linear resilient members 5a may be any combination of linear resilient members 5a having different ratios of expansion and contraction. The resilient members 5 are not limited to the linear shape, and a sheet-shaped resilient member having a number of holes or cutouts to provide a predetermined air permeability may also be used.

### REFERENCE SIGNS LIST

1 clothing
2 first fiber sheet
3 second fiber sheet
4 third fiber sheet
5 resilient member
10 disposable pants
11 outer sheet
12 waist-worn portion
13 leg portion
15 multi-ply sheet
16 joint portion
20, 30, 40 reversing device

1. A clothing manufacturing apparatus comprising:
a first member defining a space to allow lower body clothing to pass through from one end side to another end side; and
reversing means configured to reverse the lower body clothing inside out by passing the lower body clothing covering the one end side of the first member through the space to the other end side.

2. The clothing manufacturing apparatus according to claim 1, wherein the reversing means reverses the lower body clothing inside out and separates the lower body clothing from the first member.

3. The clothing manufacturing apparatus according to claim 1, wherein the reversing means is pushing means configured to mechanically push a crotch portion of the lower body clothing from outside to inside of the first member.

4. The clothing manufacturing apparatus according to claim 1, wherein the reversing means is injecting means configured to inject compressed air against the crotch portion of the lower body clothing.

5. The clothing manufacturing apparatus according to claim 1, wherein the reversing means is sucking means configured to suck the crotch portion of the lower body clothing.

6. The clothing manufacturing apparatus according to claim 1,
wherein the first member is a cylindrical member having openings at both ends, and
a length of an outer periphery of the cylindrical member matches a length of an inner periphery of a waist opening having a resilient member of the lower body clothing in a state in which the waist opening is opened against a resilient force of the resilient member.

7. The clothing manufacturing apparatus according to claim 1, wherein a surface of the first member has a rough surface configured to provide resistance against a movement of the lower body clothing.

8. A clothing manufacturing method comprising:
covering lower body clothing over one end side of a first member defining a space to allow lower body clothing to pass through from the one end side to another end side; and
reversing the lower body clothing inside out while passing the lower body clothing covering the one end side of the first member through the space to the other end side.

9. The clothing manufacturing apparatus according to claim 1, wherein the reversing means reverses the lower body clothing inside out by passing the lower body clothing in a state in which the first member is inserted through a waist opening of the lower body clothing and a crotch portion of the lower body clothing covers the space from the one end side of the first member through the space from the crotch portion.
